(19) United States
(12) Patent Application Publication
(10) Pub. No.: US 2011/0151100 A1
(43) Pub. Date: Jun. 23, 2011

(54) Green Process for Production of Biosurfactants or Biopolymers through Waste Oil Utilization

(76) Inventor: Satyanarayana Ganti, Dayton, NJ (US)

(21) Appl. No.: 12/642,785

(22) Filed: Dec. 19, 2009

(51) Int. Cl. A23L 2/00; A23J 1/00; C12P 21/06; A23K 1/00

(52) U.S. Cl. 426/590; 426/656; 435/70.1

(57) ABSTRACT

The proposed patent describes a commercially viable process for production of biosurfactants and biopolymers by employing environmentally safe utilization of waste oil after recycling or during the early stage recycling. The process described eliminates use of strong solvents or acids during the process as has been suggested by other inventors. The entire process is achieved without addition of any nutrients during fermentation and there will not be any waste produced for disposal. The end products are natural and biodegradable and have many useful industrial and commercial applications. The invention is one of the many application of patented biodispersion technology (U.S. Pat. No. 6,267,888). The invention describes production of biosurfactant from both marine water and fresh water as a source for fermentation. Three byproducts are produced during the process namely 1. Oil for recycling/blending, 2. Process water for production of biosurfactants and 3. Bacterial biomass for use as nutritive supplement or source of protein and amino acids. A yield of 0.05% was recorded in the studies.

### FIELD OF INVENTION

The invention describes a method employing environmentally safe process for utilization of waste oil for commercial production of biosurfactants and biopolymers through a green process using waste oil without employing strong solvents or acids. The end products are natural and biodegradable and have many useful industrial and commercial applications. This invention is one of the many potential commercial applications of patented Biodispersion process (U.S. Pat. No. 6,267,888).

### BACKGROUND OF THE INVENTION

Biodispersion process has been used in developing many bioremediation products that are currently used to cleanup contamination in soils, water and in reducing Volatile Organic Carbon (VOC) levels after a fuel oil spill in indoor air in an environmentally safe manner. There is no waste for disposal after cleanup. The following list captures a few salient accomplishments of our technology. More detail is available on our website: http://www.sarvabioremed.com
SpiliRemed (Marine)® has been included by the US EPA as a bioremediation agent in the National Contingency Product List for cleanup of marine open water oil spills.
VaporRemed® has been used to eliminate fuel oil fumes almost instantly and permanently and has helped in reducing the levels of toxic fuel oil fumes in homes and industrial establishments and improving indoor air quality.
SpillRemed (Industrial) has been used for cleanup of oily waste water at ports, airports and oil spills contained in berm before release of water into waste streams.

The current invention focuses on the application of the above three products based on the successful application on the field.

It is well-documented that during degradation of hydrocarbons by bacteria, certain surface active substances popularly known as biosurfactants are produced extracelluarly and some within the cell membrane. These compounds reduce the surface tension between oil and water and thus promoting transfer of hydrocarbon molecules across the cell wall for final consumption by the bacteria. Gutnik et al., (1980) have observed the foregoing. “Notwithstanding the many publications on the subject, however, microbially induced emulsification of oil is poorly understood from both mechanistic and teleological point of view. Microorganisms can utilize crude oil as a substrate for growth with or without concomitant oil emulsification. Where emulsification has occurred because of the production of extra cellular emulsifying agents, in general the preparations have not been purified sufficiently to identify the active components. In sum, none of these extra cellular bioemulsifiers has been well characterized and very little is known about their chemical properties, mode of action or biological function”. The observation is still true even after more than twenty years later. The current patent focuses on the production of extra-cellular emulsifying agents produced during the 21 day life-cycle of bioremediation of oil.

### SUMMARY OF INVENTION

The following definitions are used for various terms throughout the patent application.

Environment-friendly green process; A process using low quantities of harmful chemicals and solvents

Biodegradable products: Products produced by microbes or natural products are defined as biodegradable

Biosurfactants: Surface active compounds produced by the microbes during utilization of oil

Biopolymers: Organic polymeric compounds other than surfactants produced during fermentation through extracellular activity of bacteria.

Bioremediation products: Products developed based on my patented biodispersion process for remediation of oils contaminating soil, water and air in the form of fumes

Waste oil: Oil remaining after repeated use and cannot be used further

Bacterial products: Products containing bacteria as single species or in consortium

Indigenous bacterial population: Isolation of oil degrading bacteria from the local environment

Microbes: A general term to describe bacteria and yeasts and fungi

Industrial fermentation: Breakdown of organic substances and re-assembly into other substances

The invention describes a method employing environmentally safe process for utilization of waste oil for commercial production of biosurfactants and biopolymers through a green process without employing strong solvents or acids. Waste oils are utilized by a consortium of bacteria in bioremediation products developed on the basis of patent titled, “Biodispersion as a method for removal of hydrocarbon oil from marine aqueous environments (U.S. Pat. No. 6,267,888).” The source oil is the residual sludge remaining after recycling of the oil, waste oil remaining in the refinery process, waste diesel, aviation fuel, No. 2 heating oil, No. 6 bunker fuel, biodiesel or any other hydrocarbon that produces a waste that would be disposed in a landfill increasing the scope of groundwater pollution.

The process is achieved by mixing or fermenting waste oil with water in the ratio of 10 parts oil to 89 parts water and 1 part (v/v) of the bioremediation product such as VaporRemed®, SpillRemed (Marine)®, SpillRemed (Industrial)® or any other product developed on the basis of biodispersion process. The microbes in the bioremediation product may be in the form of a consortium of many bacteria, in the form of a single bacterial species or single species of yeasts that can be used for utilization of waste oils. The mixing/fermentation is carried out in mixing tanks ranging in size from 10 gallons to 100 gallons and can be set up at any industrial facility. The ratio of waste oil to water can be increased from ideally 10:90 (v/v) up to 50:50 (v/v) for greater yield of biosurfactants.

There is no addition of any nutrients during the this industrial fermentation unlike all other known processes for production of biosurfactants. Period of fermentation is 96 hours and can be changed from 24 hours to 240 hours required. The amount of bioremediation product is also variable and though for most processes the ratio of oil to Product is 10:1 the ratio of product could also be changed as per the process requirement. The type and quantity of bioremediation product used is based on the properties of waste oil to be consumed by bacteria. In most cases normal chlorine-free spring water or ground water is used but the process can also be carried out in filtered sea water.

The entire process of mixing or fermentation is shown in the process diagrams Once the fermentation is, completed the mixture is processed through a centrifuge to separate oil, process water and bacterial biomass. Tubular centrifuge has been used for the prototype application, but the separation can be achieved using any type of commercially available centrifuge that can effectively separate oil. Process water and residual biomass as a pellet. Each of the two phases namely water and biomass is pooled further for isolation of biosurfactants and protein mixtures. The residual oil after separation is evaluated for its suitability for reuse after blending for commercial application or is re-employed as source oil for fermentation till the oil is completely consumed through bioremediation. The process water is processed to isolate the biosurfactant or biopolymer by evaporating the aqueous phase till dry. The dried product is then chemically analyzed for the nature and properties. The product may be purified further, if necessary, prior to use. The process water is collected through distillation during the process of evaporation using roto-evaporators in order to conserve the resources and reuse the same water in repeat cycles with minimum loss. The biomass collected is a rich source of protein and is either used as a feed for poultry or after further processing used as a source for nutrient supplements like amino acids. The invention proposes to use the waste oil as a primary resource to produce many products for industrial application and replace chemicals damaging environment with biodegradable natural substitutes.

### BRIEF DESCRIPTION OF FIGURES

FIG. 1 depicts the industrial process for production of biosurfactants from waste oil. The process uses non-chlorinated, non-saline water.

FIG. 2 shows the steps involved in the production of biosurfactants from waste oil using salt water.

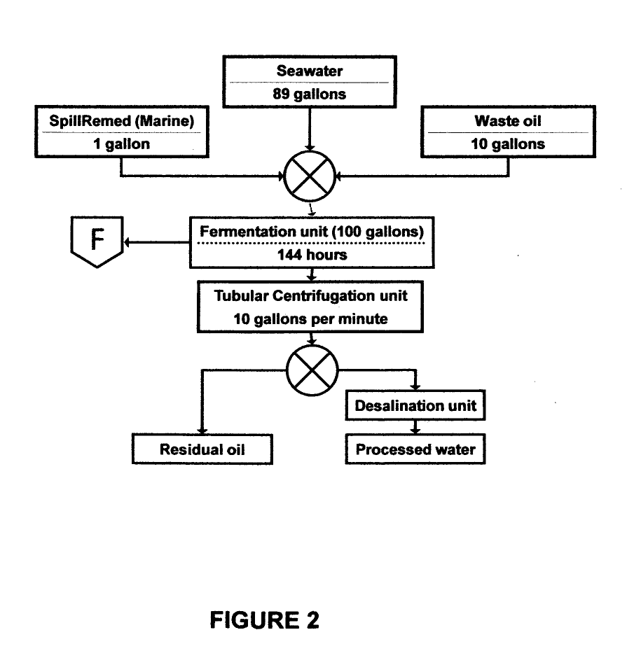

FIG. 3 depicts the key steps for the processing for biosurfactants.

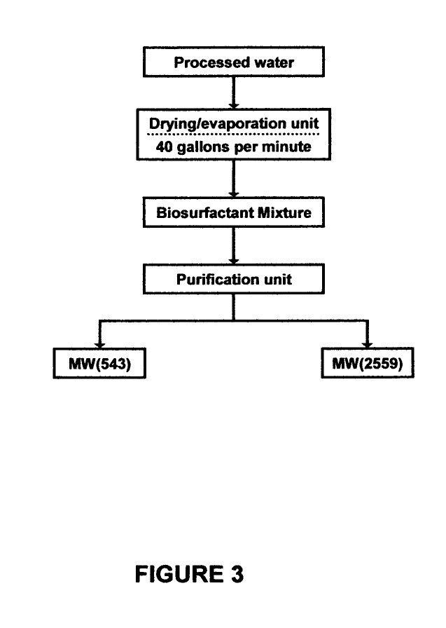

FIG. 4 is a flow chart of the processing of recovered Oil after separation from water

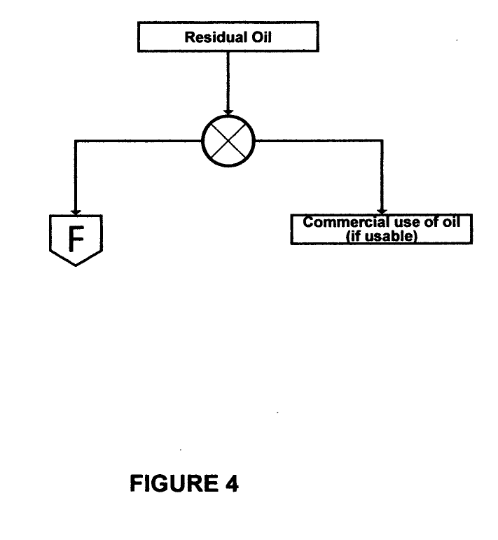

FIG. 5 shows the steps involved with the production of byproducts of biomass

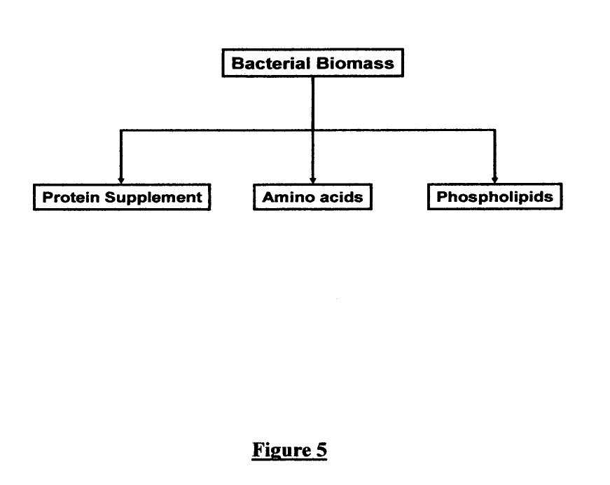

### DETAILED DESCRIPTION

### Process Description

In the present invention describes two novel features: 1) there is no addition of nutrients during fermentation and 2) a consortium of oil-consuming bacteria are used as the source. Both these features improve the commercial production of biosurfactants and increase the robustness of the overall process. The invention extends the application of bioremediation of waste oil to produce beneficial byproducts. In contrast, the current state-of-the-art processes on the production of biosurfactants use inorganic salts and organic nutrient supplements during a fermentation process using a single species and or genetically modified forms as the source of biosurfactants.

The three products mentioned above have shown effective bioremediation of oil when present in water and therefore are selected for studies on the production of biosurfactants in an oil and water mixture. The ratio of bioremediation product to oil and water is 1:10:89. For example, 10 gallons of waste oil is fermented in 89 gallons of water free from chlorine, toxic chemicals or organic contamination with 1 gallon of any of the three products stated above are fermented in a 100 gallon fermentation tank. Fermentation using SpillRemed (Marine) is carried out in marine/sea water. In the present invention for manufacture biosurfactants the process lifecycle is reduced to 96 hours or 180 hours for faster and higher production of biosurfactants and is not allowed to continue till complete consumption of oil.

Separation of oil from water after the fermentation is achieved using tubular centrifuge with a speed of 8,000 RPM to 14,000 RPM with at least two outlets one to recover oil and the second outlet for water free from oil and biomass is collected. Use of other effective or cheaper methods to separate the oil from the water is not precluded from this invention, as in our opinion, that part is not the core property of this invention and in fact would allow us to improve the efficiency of our process with added improvements in separation technology. Finally following three products result after centrifugation namely
Residual oil
Process water containing biosurfactants and biopolymer
Bacterial biomass

The residual oil can be used either for re-refining and blending for improved property or recycled back into the fermentation tank for further production. In cases where the oil is heavier in nature for example bunker fuel, furnace fuel oil, No. 6 fuel oil or Naval Special Fuel Oil (NSFO) is used as a source for biosurfactant production, the resultant oil could be recycled as a new product with proper blending.

The process water free from oil is then filtered through 0.22 micron filters to remove residual bacteria or biomass. The process water is then evaporated to dryness using a forced air drying, spray drying or a roto-evaporator unit. The dried powder can be subsequently used as a biosurfactant concentrate with applications in, but not limited to pharmaceutical formulations, healthcare products, skin care products, soil washings, and cleanup of contaminated ground water, enhanced recovery of oil from low yielding oil wells etc.

The biomass is collected from the centrifuge and is then used for further processing for use as animal feed, protein supplement or for production of amino acids based on the requirements of the industry.

The process described in the invention is inherently environmentally safe and since the process does not involve use of strong organic solvents the process does not add to any pollution footprint of its own.

The process has shown a yield of 0.05% in bench scale studies and the efficiency of this process can be enhanced to improve the yield. Diagram 1 outlines a general process scaled to a 100 gallon fermenter and could be scaled up to larger capacities for larger production values.

### Process Time (Optimal Value: 144 Hours)

In our laboratory studies we recorded that there is a reduction of almost 30% of the source oil within 144 hours. The mixture forms a strong emulsion of oil in water through the process of biodispersion and the bacterial population seems to reach its peak at 144 hours and therefore have used 144 hours as the initial cut-off point for the proposed process. However, it is possible to reduce or increase the process time as per the requirement and could be anywhere from 24 hours to 21 days depending on the volume and quality of the yield required.

### Inoculums Ratio (Optimal Value—1 Part Product: 10 Parts Oil)

We recommend that our customers use our products in the ratio of 1 part of our product to 10 parts of oil for bioremediation of contaminating oil as the most cost-effective ratio for complete bioremediation of oil. Keeping this in mind the ratio for the invention is set to the same optimal value. However, we have observed in field that increasing the ratio helps either to speed up the bioremediation or work on heavier forms of oil. Thus when used on thinner oils such as heating oil or bilge oil the increased ratio help speed up process and in case of heavier oils such as bunker fuel, this process can help in reducing the viscosity of oil and oil is thinned sufficiently to be a part of the re-refining process.

### Example 1

### Population Growth of Bacterial Consortium

SpillRemed (Marine)® is a consortium of oil eating bacteria isolated from the sea water samples at Prince Sound Williams in Alaska rich in the population of oil degrading bacteria (ODB). SpillRemed (Marine)® has also been used for treating oily bilge water under the trade name BilgeRemed for oily waste water holding tank and for cleanup of open water oil spills and cleanup of oily bilge water on board ships either during sailing or during berthed at the shore. In the present example, we present the data about the consortium of the bacteria that constitute SpillRemed (Marine) and the population growth of the bacteria in oily sludge. The consortium of bacteria has been shown to grow geometrically as is to be expected from a population growing in favorable conditions (see Table 1). The consortium of bacteria in SpillRemed (Marine)® is comprised of:
Pseudomonas pseudoalkaligenes
Phenylobacterium immobile
Stenotrophomonas maltophilia,
Gluconobacter cerinus
Agrobacterium radiobacter
Pseudomonas alcaligenes

TABLE 1
Bacterial growth of SpillRemed (Marine) when added to oily sludge
Growth of Bacteria in Sludge received from “Legend of the Seas”

| Date of sampl | Time (Days) | Colonies of bacteria per ml. |
|---|---|---|
| Mar. 18, 2004 | 0.00 | 500,000 |
| Mar. 19, 2004 | 1 | 7,300,000 |
| Mar. 22, 2004 | 4 | 940,000,000 |
| Mar. 24, 2004 | 6 | 12,100,000,000 |
| Apr. 1, 2004 | 14 | 790,000,000 |

The data above shows that bacterial population increases in the presence of oil and the population reached a value of 12.1 billion on 6^{th} day indicating a healthy biomass suitable for optimum yield of both the biosurfactants and the biomass. The process of isolating biosurfactants remains generally same as described above but in this case sea water is used instead of fresh water for fermentation. The final product is therefore was desalted to remove the salts contributing to the weight. SpillRemed (Marine)® can be used in recycling of waste oils generated in harbors, ports or collected after a major oil spill in the sea. The large biomass could thus be used for any other applications mentioned above.

### Example 2

### Biopolymer Extraction Results

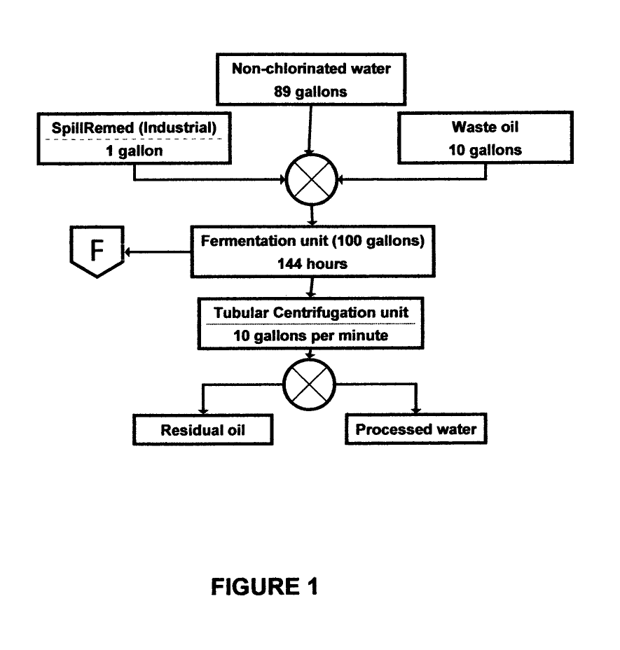

SpillRemed (Industrial) is a lower salinity version as compared to SpillRemed (Marine) and has been used for cleanup of oils and greases released during metal recycling or release of fuels in lagoons or in contained berm. In the present example, SpillRemed (Industrial) was processed through a tubular centrifuge and the resultant water free from oil and bacteria and was subjected to analysis of presence of any Biosurfactants by processing through the Size Exclusion Chromatography and it was found that the compound was a mixture of two molecules one at 543 Daltons (identified as M 543) and the second molecule at 2559 Daltons (identified as M2559) as seen in the FIG. 1 below and the yield of biosurfactant was determined to be 0.05%.

### Example 3

### Comparison of VaporRemed with Triton X for Reducing Interfacial Tension (IFT)

VaporRemed is a freeze-resistant version of SpillRemed (Industrial) and was used to measure the reduction of Interfacial Tension (IFT). A test was performed using VaporRemed and it was found that VaporRemed reduced the IFT but the effect was relatively short when compared to Triton X. This supported the fact that bioremediation products developed on the basis of biodispersion process produced surface active compounds.

### Example 4

### Biomass Analysis of SpillRemed (Industrial)

Analysis of biomass showed that it contained 34.2% proteins, 24.45% carbohydrates, and 18.12% lipids. Based on the high protein content in the biomass it could be used as animal feed or for use in poultry feed and could be considered as food supplement for consumption of human being Amino acid analysis indicated that the biomass was rich in the following compounds and could be considered a source for each of these amino acids. Analysis of amino acids is given in Table 2.

TABLE 2
Showing concentrations of individual amino acids in biomass

| Compound | Percent |
|---|---|
| Glutamine | 11.55 |
| Aspargine | 11.16 |
| Leucine | 9.05 |
| Lysine | 8.13 |

### Example 5

### Increased Inoculums Ratio on the Process

A 26,000 gallon oil-waste water unit at an oil-recycling center was treated using SpillRemed (Industrial) to remove the oil and discharge water free from oil within a 48 hour timeline. The requirement in this example was not complete bio-remediation, rather a uniform dispersion and the recommend ratio of 1 part product: 10 parts oil would take about 96 hours to achieve dispersion along the entire water column. As the time allowed for the process was shortened by half, the ratio was increased to 3 parts product: 10 parts oil and at the end of 48 hours the process reduced oil by 40% suggesting that it is possible to obtain higher yields of biosurfactant in a short time cycle using higher ratios as mentioned earlier and of course general observations would also allow us to reach a similar conclusions. The analysis that has not been performed is to find an upper limit for the ratio.

### Example 6

### Use of Higher Volumes of Source Oil

The ratio of water to oil is some times higher in oily bilge water on board ships and it is reported to be as high as 30% of the total volume. SpillRemed (Marine)® has been found to effectively reduce this mixture of oils and greases in less than 3 weeks time as has been found during our field trials on both actively sailing ships and ships berthed at harbors. Increase in ratio of oil can be used for improved recovery of recyclable oils and higher yield of biosurfactants.

1-21. (canceled)

22. An environment-friendly green process for the production of natural and biodegradable products by utilization of waste oils through using (you are limiting yourself) bioremediation products developed on the basis of patented biodispersion process comprising of following steps:
i) industrial fermentation of bioremediation product, oil and water in a suitable container;
ii) the said mixture is agitated using fixed impellers or any other method of agitation in suitable container for a fixed period of time;
iii) the mixture is agitated under constant temperature conditions 32 C for optimum yield (vague term)
iii) separation of oil, processed water and biomass by centrifugation;
iv) drying of processed water isolate biosurfactant as a crystalline residue; and
v) purification of active ingredient based on molecular weight (be specific).
vi) Reutilization of useful fractions of separated oil where possible particularly for dense oils like No. 6 oil as some oils are known to become thinner due to activities of bacteria in the bioremediation product as seen in our field studies.
vii) A nutritional supplement comprising protein isolated from the biomass produced during the industrial fermentation.

23. The process as in claim 22 wherein fermentation is carried out using waste oil selected from a group consisting of mixture of oils remaining after recycling of waste oil, waste oil from refinery waste, sludge remaining after recycling oil, waste remaining in the bulk storage tanks of diesel, No. 2 fuel oil, aviation fuel, No. 6 Bunker fuel, transformer oil, Naval Special Fuel Oil (NSFO), biodiesel, ships bilge oil, oil collected after a spill and oil from other hydrocarbon sources and natural vegetable oils.

24. The process as in claim 22 wherein the natural and biodegradation products are identified as biosurfactant, biopolymer and biomass.

25. The process in claim 22 wherein the industrial fermentation is initiated (this terminology should be part of summary of invention) by commercially available products like SpiliRemed (Industrial), VaporRemed®, SpillRemed (Marine)® and any other commercially available product (this is indefinite) developed on the basis of patented biodispersion process.

26. The seed for the fermentation in claim 25, wherein new bioremediation product is developed from a group of microbes consisting of aerobic bacteria, anaerobic bacteria, genetically modified aerobic bacteria, genetically modified anaerobic bacteria, yeast, fungi and mixture thereof.

27. The process as in claim 22 wherein the container is selected from commercially available chemical reaction vessels, a stainless steel fermenter, a plastic fermenter, epoxy-coated stainless steel fermenter, or container of other suitable inert non corrosive material in various sizes from 10 gallons to 1000 gallons fitted with an agitator and mixer and with pressured air, oxygen or nitrogen supply.

28. A process utilizing waste oils for the production of natural and biodegradable products in claim 22 wherein the water used is chlorine free spring water and chlorine free sea water.

29. A industrial fermentation (define it in the summary of invention) as in claim 22 wherein the ratio of amounts of waste oil to the seed is 1:1 volume/volume, preferably 5:1 volume/volume and most preferably 10:1 volume/volume.

30. A industrial fermentation process as in claim 22 wherein the ratios of amounts of water to waste oil is 50:50 volume/volume, 70:30 volume/volume, preferably 80:20 volume/volume and most preferably 90:10 volume/volume.

31. The process as in claim 26 wherein the aerobic? bacteria selected from the group consisting of. Pseudomonas, Phenylobacterium, Stenotrophomonas, Gluconobacter, Agrobacterium, Vibrio, Acinetobacter, Micrococcus, or other naturally available oil degrading bacteria.

32. The bacteria as in claim 31, wherein said species or strain of bacteria is Pseudomonas pseudoalkaligenes, Phenylobacterium immobile, Stenotrophomonas maltophilia, Gluconobacter cerinus, Agrobacterium radiobacter or Pseudomonas alcaligenes. Pseudomonas aeruginosa, and Citrobacter freundii, other indigenously available species and mixtures thereof.

33. A microbial fermentation process as in claim 22 wherein the centrifugation is achieved using tubular centrifuge, high speed refrigerated centrifuge or any separation mechanism to separate oil from water and collect bacterial biomass as a pellet or a paste. (indefinite terminology).

34. A microbial fermentation process as in claim 34 wherein process water is evaporated to dryness using evaporator vessels under vacuum to collect the dried crystalline powder of biosurfactant.

35. A process as in claim 22 wherein the active products are purified using a process from the group consisting of. dialysis, size exclusion chromatography, acid or alkali hydrolysis or any other available methods for isolation and purification (it is not in the summary of invention).

36. The biomass in claim 22 (something is missing here) obtained after centrifugation of fermentation medium is used after drying for feed, sonication of cells to break cell walls and collect the cell material; and for manufacturing of supplements from a group of biscuits, cookies, high protein liquid nutrient diets.

37. The biomass in claim 37 is used for poultry feed or animal feed after drying (can we really claim it? It can have many applications which can be described in the specifications).
